(19) United States
(12) Patent Application Publication
(10) Pub. No.: US 2017/0281541 A1
(43) Pub. Date: Oct. 5, 2017

(54) LIPOSOME-BASED MUCUS-PENETRATING PARTICLES FOR MUCOSAL DELIVERY

(71) Applicants: The Johns Hopkins University, Baltimore, MD (US); Kennedy Krieger Institute, Inc., Baltimore, MD (US)

(72) Inventors: Tao Yu, Baltimore, MD (US); Kannie W. Y. Chan, Baltimore, MD (US); Ming Yang, Towson, MD (US); Michael T. McMahon, Columbia, MD (US); Justin Hanes, Baltimore, MD (US)

(21) Appl. No.: 15/509,061

(22) PCT Filed: Sep. 1, 2015
(86) PCT No.: PCT/US2015/047931

§ 371 Date: Mar. 6, 2017

Related U.S. Application Data
(60) Provisional application No. 62/046,540, filed on Sep. 5, 2014.

(51) Int. Cl. A61K 9/127; A61K 31/515; A61K 9/00

(52) U.S. Cl. CPC A61K 9/1271; A61K 9/0048; A61K 31/515; A61K 9/0034

(57) ABSTRACT

Liposome-based mucus-penetrating particles (MPP) capable of loading hydrophilic agents including therapeutic, prophylactic and diagnostic agents such as the diaCEST MRI contrast agent barbituric acid (BA) were evaluated to determine how to optimize delivery. Polyethylene glycol (PEG)-coated liposomes containing ≧7 mol % PEG diffused only approximately 10-fold slower in human cervicovaginal mucus (CVM) compared to their theoretical speeds in water. 7 mol %-PEG liposomes provided improved vaginal distribution compared to 0 and 3 mol %-PEG liposomes.

### CROSS REFERENCE TO RELATED APPLICATIONS

This application claims benefit of and priority to U.S. Provisional Patent Application No. 62/046,540 filed on Sep. 5, 2014, and where permissible is hereby incorporated by reference in its entirety.

### STATEMENT REGARDING FEDERALLY SPONSORED RESEARCH OR DEVELOPMENT

This invention was made with Government Support under NIH grants R01EB015031, R01EB015032, and S10RR028955 by the National Institutes of Health. The Government has certain rights in the invention.

### BACKGROUND OF THE INVENTION

Localized delivery of therapeutics via biodegradable liposomes often provides advantages over systemic drug administration, including reduced systemic side effects and controlled drug levels at target sites. However, controlled drug delivery at mucosal surfaces has been limited by the presence of the protective mucus layer. The same issues apply to diagnostics.

Mucus is a viscoelastic gel that coats all exposed epithelial surfaces not covered by skin, such as respiratory, gastrointestinal, nasopharyngeal, and female reproductive tracts, and the surface of eye. Mucus efficiently traps conventional particulate drug delivery systems via steric and/or adhesive interactions. As a result of mucus turnover, most therapeutics delivered locally to mucosal surfaces suffer from poor retention and distribution, which limits their efficacy.

The surface of the vagina is highly folded to accommodate expansion during intercourse and childbirth; these folds, or “rugae,” are normally collapsed by intra-abdominal pressure, hindering drug delivery to the folded surfaces. For truly effective prevention and treatment, sustained drug concentrations must be delivered to, and maintained over the entire susceptible surface. Failure to achieve adequate distribution over the entire vaginal epithelium is a documented failure mode of vaginal microbicides.

Another significant barrier to effective drug delivery to the vagina is the viscoelastic layer of mucus secreted by the endocervix that coats the vaginal epithelium. Mucus efficiently traps foreign particles and particulates by both steric and adhesive mechanisms, facilitating rapid clearance.

Although the use of mucoadhesive dosage forms has been proposed for increasing residence time in the vagina, mucus clearance occurs rapidly (on the order of minutes to hours), limiting the residence time of mucoadhesive systems.

Mucosal epithelia use osmotic gradients to cause fluid absorption and secretion. Vaginal products have traditionally been made with hypertonic formulations, including yeast infection treatments, most sexual lubricants such as KY® warming gel, and gels designed for preventing sexually transmitted infections such as HIV. Hypertonic formulations cause rapid, osmotically-driven secretion of fluid into the vagina, and this causes an immediate increase in fluid leakage from the vagina at a rate proportional to the hypertonicity of the formulation. Moreover, recent investigations of candidate vaginal and rectal microbicides both in animal models and in humans have revealed that hypertonic formulations cause toxic effects that can increase susceptibility to infections. The first successful microbicide trial for HIV prevention found that the antiretroviral drug, tenofovir, delivered in a vaginal gel, provided partial protection. Unfortunately, the gel formulation was highly hypertonic, leading investigators in the most recent clinical trial of tenofovir to reduce the concentration of glycerol to reduce toxicity. However, the concentration was not reduced, and the formulation is still significantly hypertonic. There appears to be no evidence to justify hypertonic formulations for vaginal drug delivery, since in addition to the documented toxic effects, hypertonic formulations cause rapid osmotically-driven secretion of vaginal fluid, fluid flow that opposes the delivery of drugs to the epithelium. This lack of justification has been ignored by both investigators and manufacturers of vaginal products, the only evident exception being sexual lubricants intended to support fertilization. These products are formulated to be isotonic (the osmolality is equivalent to that of plasma) to help maintain viability of sperm.

Drug and gene carrying liposomes delivered to mucus-covered cells in the eyes, nose, lungs, gastrointestinal tract, and female reproductive tract must achieve uniform distribution in order to maximally treat or protect these surfaces. However, the highly viscoelastic (i.e., viscous and solid-like in nature) and adhesive mucus layer can slow or completely immobilize particles, and thereby prevent them from spreading over the mucosal surface. In addition, some mucosal surfaces, such as those of the mouth, stomach, intestines, colon, and vagina, exhibit highly folded epithelial surfaces that are inaccessible to conventional muco-adhesive particles and also to many small molecule drugs and therapeutics. Without maximal distribution with penetration into these deep recesses, much of the epithelium is left susceptible and/or untreated. Additionally, penetration into the folds, presumably containing a much more slowly cleared mucus layer, allows for increased residence time at the epithelial surface.

For drug or gene delivery applications, therapeutic particles must be able to 1) achieve uniform distribution over the mucosal surface of interest, as well as 2) cross the mucus barrier efficiently to avoid rapid mucus clearance and ensure effective delivery of their therapeutic payload to underlying cells (das Neves J & Bahia M F Int J Pharm 318, 1-14 (2006); Lai et al. Adv Drug Deliver Rev 61, 158-171 (2009); Ensign et al. Sc. Transl Med 4, 138ral79, 1-10 (2012); Eyles et al. J Pharm Pharmacol 47, 561-565 (1995)).

Biodegradable liposomes that penetrate deep into the mucus barrier can provide improved drug distribution, retention and efficacy at mucosal surfaces. Dense surface coats of low molecular weight polyethylene glycol (PEG) allow liposomes to rapidly penetrate through highly viscoelastic human and animal mucus secretions. The hydrophilic and bioinert PEG coating effectively minimizes adhesive interactions between liposomes and mucus constituents. Biodegradable mucus-penetrating particles (MPPs) have been prepared by physical adsorption of certain PLURONICs, such as F127, onto pre-fabricated mucoadhesive particles.

Mucosal drug delivery via nano-carriers holds potential to improve detection and treatment of numerous diseases.^{1, 2} For efficient mucosal delivery, nano-carriers must first bypass the highly protective mucus linings that rapidly remove most foreign particles from the mucosae. To overcome the mucus barrier, we have previously developed polymer- and pure drug-based nanoparticulates that possess dense coatings with polyethylene glycol (PEG) that effectively avoid mucoadhesion, thus allowing rapid penetration through mucus. As a result, these mucus-penetrating particles (MPP) provide more uniform distribution and sustained delivery of therapeutics at various mucosal sites.

Liposomes were the first nano-carrier system to be developed and translated for clinical use. Although liposomal systems have been explored for mucosal delivery, there has not been a focus on directly observing the interactions of liposomal formulations with mucus, and how these interactions impact mucosal distribution.

Therefore, it is an object of the invention to provide formulations for rapid and uniform particulate delivery of a wide range of drugs and/or imaging agents to mucosal covered epithelial surfaces with minimal toxicity to the epithelium.

### SUMMARY OF THE INVENTION

Liposome-based mucus-penetrating particles (MPP) capable of loading hydrophilic agents including therapeutic, prophylactic and diagnostic agents such as the diaCEST MRI contrast agent barbituric acid (BA) were evaluated to determine how to optimize delivery. Polyethylene glycol (PEG)-coated liposomes containing ≧7 mol % PEG diffused only approximately 10-fold slower in human cervicovaginal mucus (CVM) compared to their theoretical speeds in water. 7 mol %-PEG liposomes provided improved vaginal distribution compared to 0 and 3 mol %-PEG liposomes. Liposome-based mucus-penetrating particles (MPP) capable of loading hydrophilic agents including therapeutic, prophylactic and diagnostic agents such as the diaCEST MRI contrast agent barbituric acid (BA) were evaluated to determine how to optimize delivery. Polyethylene glycol (PEG)-coated liposomes containing ≧7 mol % PEG diffused only approximately 10-fold slower in human cervicovaginal mucus (CVM) compared to their theoretical speeds in water. 7 mol %-PEG liposomes provided improved vaginal distribution compared to 0 and 3 mol %-PEG liposomes. However, increasing PEG content to approximately 12 mol % compromised BA loading and vaginal distribution, indicating that PEG content must be optimized to maintain drug loading and in vivo stability. Non-invasive diaCEST MRI illustrated uniform vaginal coverage and longer retention of BA-loaded 7 mol %-PEG liposomes compared to unencapsulated BA.

Liposomal particles can be mucus-penetrating or mucoadhesive. The surface PEG density has to be within an optimal range to achieve the best mucus-penetration features. In terms of ex vivo mobility in human cervicovaginal mucus, PEGylated liposomes (even as low as 3 mol %) move faster than non-PEGylated liposomes; higher PEG surface density leads to slightly improved mobility. In terms of in vivo distribution in mouse vagina, much higher PEG molar fraction (i.e., 12 mol % or higher) caused unexpectedly non-uniform distribution of the liposomes, implying their weak stability in vivo. The liposomal drug loading is also compromised by high PEG molar fraction.

In the preferred embodiment, the PEG is between 3 and 10 mol % of the liposomes. The optimal amount may be affected by the type of lipids used in the formulation. In the studies in the examples, DSPC was used as the primary lipid, which is neutrally charged. The PEG density may need to be increased for liposomes composed of non-neutrally charged lipids.

### BRIEF DESCRIPTION OF THE DRAWINGS

FIGS. 1A-1D. Mobility of PEGylated and non-PEGylated DSPC liposomes 0 h or 3 h post addition to CVM. FIG. 1A are representative liposome trajectories over 1 s. FIG. 1B and FIG. 1C are graphs of the <MSD> (μm²) as a function of time (seconds). FIG. 1D are distribution graphs (% particles) of the logarithms of individual liposome MSD.

FIG. 2 is a graph of the Distribution of red fluorescent BA-loaded liposomes on flattened mouse vaginal tissue, as a function of different PEGylation levels (mol %) to Variance-to-mean ratio of fluorescence intensity (Lower values indicate increased uniformity).

FIGS. 3A and 3B are graphs of intravaginally administered BA-loaded liposomal MPP and unencapsulated BA via MRI in mice, FIG. 3A showing relative MTR_{asym} over time and FIG. 3B showing a histogram of pixelated MTR_{asym} at 90 min.

FIG. 4 is a graph of the retention of BA and the liposomal CEST contrast for 7 mol %-PEG DSPC liposomes in vitro. (n=4 independent measurements), % of BA retained over time (hours).

### DETAILED DESCRIPTION OF THE INVENTION

### I. Definitions

“Active Agent,” as used herein, refers to a physiologically or pharmacologically active substance that acts locally and/or systemically in the body. An active agent is a substance that is administered to a patient for the treatment (e.g., therapeutic agent), prevention (e.g., prophylactic agent), or diagnosis (e.g., diagnostic agent) of a disease or disorder. “Ophthalmic Drug” or “Ophthalmic Active Agent”, as used herein, refers to an agent that is administered to a patient to alleviate, delay onset of, or prevent one or more symptoms of a disease or disorder of the eye, or diagnostic agent useful for imaging or otherwise assessing the eye.

“Effective amount” or “therapeutically effective amount,” as used herein, refers to an amount of polymeric liposome effective to alleviate, delay onset of, or prevent one or more symptoms, particularly of a disease or disorder of the eye. In the case of age-related macular degeneration, the effective amount of the polymeric liposome delays, reduces, or prevents vision loss in a patient. “Biocompatible” and “biologically compatible,” as used herein, generally refer to materials that are, along with any metabolites or degradation products thereof, generally non-toxic to the recipient, and do not cause any significant adverse effects to the recipient. Generally speaking, biocompatible materials are materials which do not elicit a significant inflammatory or immune response when administered to a patient.

“Biodegradable Polymer,” as used herein, generally refers to a polymer that will degrade or erode by enzymatic action and/or hydrolysis under physiologic conditions to smaller units or chemical species that are capable of being metabolized, eliminated, or excreted by the subject. The degradation time is a function of polymer composition, morphology, such as porosity, particle dimensions, and environment.

“Hydrophilic,” as used herein, refers to the property of having affinity for water. For example, hydrophilic polymers (or hydrophilic polymer segments) are polymers (or polymer segments) which are primarily soluble in aqueous solutions and/or have a tendency to absorb water. In general, the more hydrophilic a polymer is, the more that polymer tends to dissolve in, mix with, or be wetted by water.

“Hydrophobic,” as used herein, refers to the property of lacking affinity for, or even repelling water. For example, the more hydrophobic a polymer (or polymer segment), the more that polymer (or polymer segment) tends to not dissolve in, not mix with, or not be wetted by water.

Hydrophilicity and hydrophobicity can be spoken of in relative terms, such as but not limited to a spectrum of hydrophilicity/hydrophobicity within a group of polymers or polymer segments. In some embodiments wherein two or more polymers are being discussed, the term “hydrophobic polymer” can be defined based on the polymer's relative hydrophobicity when compared to another, more hydrophilic polymer.

“Liposome,” as used herein, generally refers to a particle having a diameter, such as an average diameter, from about 10 nm up to but not including about 1 micron, preferably from 100 nm to about 1 micron. The particles can have any shape. Liposomes having a spherical shape are generally referred to as “nanospheres”.

“Microparticle,” as used herein, generally refers to a particle having a diameter, such as an average diameter, from about 1 micron to about 100 microns, preferably from about 1 micron to about 50 microns, more preferably from about 1 to about 30 microns. The microparticles can have any shape. Microparticles having a spherical shape are generally referred to as “microspheres”.

“Molecular weight,” as used herein, generally refers to the relative average chain length of the bulk polymer, unless otherwise specified. In practice, molecular weight can be estimated or characterized using various methods including gel permeation chromatography (GPC) or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

“Mean particle size,” as used herein, generally refers to the statistical mean particle size (diameter) of the particles in a population of particles. The diameter of an essentially spherical particle may refer to the physical or hydrodynamic diameter. The diameter of a non-spherical particle may refer preferentially to the hydrodynamic diameter. As used herein, the diameter of a non-spherical particle may refer to the largest linear distance between two points on the surface of the particle. Mean particle size can be measured using methods known in the art, such as dynamic light scattering.

“Monodisperse” and “homogeneous size distribution” are used interchangeably herein and describe a population of liposomes or microparticles where all of the particles are the same or nearly the same size. As used herein, a monodisperse distribution refers to particle distributions in which 90% or more of the distribution lies within 15% of the median particle size, more preferably within 10% of the median particle size, most preferably within 5% of the median particle size.

“Pharmaceutically Acceptable,” as used herein, refers to compounds, carriers, excipients, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

### II. Formulations

Liposomes are used as carriers for drugs and antigens because they can serve several different purposes (Storm & Crommelin, Pharmaceutical Science & Technology Today, 1, 19-31 1998). Liposome encapsulated drugs are inaccessible to metabolizing enzymes. Conversely, body components (such as erythrocytes or tissues at the injection site) are not directly exposed to the full dose of the drug. The duration of drug action can be prolonged by liposomes because of a slower release of the drug in the body. Targeted liposomes change the distribution of the drug over the body. Cells use endocytosis or phagocytosis mechanisms to take up liposomes into the cytosol. Furthermore liposomes can protect a drug against degradation (e.g. metabolic degradation). Although sometimes successful, liposomes have limitations. Liposomes not only deliver drugs to diseased tissue, but also rapidly enter the liver, spleen, kidneys and Reticuloendothelial Systems, and leak drugs while in circulation (Harris & Chess, Nature, March 2003, 2, 214-221).

Liposome membranes containing bilayer-compatible species such as poly (ethylene glycol)-linked lipids (PEG-lipid) or gangliosides are used to prepare stealth liposomes (Papahadjopoulos et al., PNAS, 88, 11460-4 1991). Stealth liposomes have a relatively long half-life in blood circulation and show an altered biodistribution in vivo. Vaage et al. (Int. J. of Cancer 51, 942-8, 1992) prepared stealth liposomes of doxorubicin and used them to treat recently implanted and well established growing primary mouse carcinomas, and to inhibit the development of spontaneous metastases from intra-mammary tumor implants. They concluded that the long circulation time of the stealth liposomes of doxorubicin formulation accounts for its superior therapeutic effectiveness. The presence of MPEG-derivatized (pegylated) lipids in the bilayers membrane of sterically stabilized liposomes effectively furnishes a steric barrier against interactions with plasma proteins and cell surface receptors that are responsible for the rapid intravascular destabilization/rupture and RES clearance seen after i.v. administration of conventional liposomes. As a result, pegylated liposomes have a prolonged circulation half-life, and the pharmacokinetics of any encapsulated agent are altered to conform to those of the liposomal carrier rather than those of the entrapped drug (Stewart et al., J. Clin. Oncol. 16, 683-691, 1998). Because the mechanism of tumor localization of pegylated liposomes is by means of extravasation through leaky blood vessels in the tumor (Northfelt et al., J. Clin. Oncol. 16, 2445-2451, 1998; Muggia et al., J. Clin. Oncol. 15, 987-993, 1997), prolonged circulation is likely to favor accumulation in the tumor by increasing the total number of passes made by the pegylated liposomes through the tumor vasculature.

A. Liposomes

Liposomes with modified surfaces have been developed with the synthetic polymer poly-(ethylene glycol) (PEG) on the surface of the liposomal carrier. These have been shown to extend blood-circulation time while reducing mononuclear phagocyte system uptake (stealth liposomes). These can be used to encapsulate active molecules, with high target efficiency and activity. Further, by synthetic modification of the terminal PEG molecule, stealth liposomes can be actively targeted with monoclonal antibodies or ligands.

Liposomes are biocompatible and biodegradable. They consist of an aqueous core entrapped by one or more bilayers composed of natural or synthetic lipids. Liposomes composed of natural phospholipids are biologically inert and weakly immunogenic, and they possess low intrinsic toxicity. Further, drugs with different lipophilicities can be encapsulated into liposomes: strongly lipophilic drugs are entrapped almost completely in the lipid bilayer, strongly hydrophilic drugs are located exclusively in the aqueous compartment, and drugs with intermediate log P easily partition between the lipid and aqueous phases, both in the bilayer and in the aqueous core. Liposomes can be classified according to their lamellarity (uni-, oligo-, and multi-lamellar vesicles), size (small, intermediate, or large) and preparation method (such as reverse phase evaporation vesicles, VETs). Unilamellar vesicles comprise one lipid bilayer and generally have diameters of 50-250 nm. They contain a large aqueous core and are preferentially used to encapsulate water-soluble drugs. Multilamellar vesicles comprise several concentric lipid bilayers in an onion-skin arrangement and have diameters of 1-5 μm. The high lipid content allows these multilamellar vesicles to passively entrap lipid-soluble drugs. Unilamellar vesicles are described herein due to the need for a small diameter of less than one micron, more preferably less than 500 nm.

Selection of the appropriate lipids for liposome composition is governed by the factors of: (1) liposome stability, (2) phase transition temperature, (3) charge, (4) non-toxicity to mammalian systems, (5) encapsulation efficiency, (6) lipid mixture characteristics. The vesicle-forming lipids preferably have two hydrocarbon chains, typically acyl chains, and a head group, either polar or nonpolar. The hydrocarbon chains may be saturated or have varying degrees of unsaturation. There are a variety of synthetic vesicle-forming lipids and naturally-occurring vesicle-forming lipids, including the sphingolipids, ether lipids, sterols, phospholipids, particularly the phosphoglycerides, and the glycolipids, such as the cerebrosides and gangliosides.

Phosphoglycerides include phospholipids such as phosphatidylcholine, phosphatidylethanolamine, phosphatidic acid, phosphatidylinositol, phosphatidylserine phosphatidylglycerol and diphosphatidylglycerol (cardiolipin), where the two hydrocarbon chains are typically between about 14-22 carbon atoms in length, and have varying degrees of unsaturation. As used herein, the abbreviation “PC” stands for phosphatidylcholine, and “PS” stand for phosphatidylserine. Lipids containing either saturated and unsaturated fatty acids are widely available to those of skill in the art. Additionally, the two hydrocarbon chains of the lipid may be symmetrical or asymmetrical. The above-described lipids and phospholipids whose acyl chains have varying lengths and degrees of saturation can be obtained commercially or prepared according to published methods.

Exemplary phosphatidylcholines include dilauroyl phophatidylcholine, dimyristoylphophatidylcholine, dipalmitoylphophatidylcholine, distearoylphophatidyl-choline, diarachidoylphophatidylcholine, dioleoylphophatidylcholine, dilinoleoyl-phophatidylcholine, dierucoylphophatidylcholine, palmitoyl-oleoyl-phophatidylcholine, egg phosphatidylcholine, myristoyl-palmitoylphosphatidylcholine, palmitoyl-myristoyl-phosphatidylcholine, myristoyl-stearoylphosphatidylcholine, palmitoyl-stearoyl-phosphatidylcholine, stearoyl-palmitoylphosphatidylcholine, stearoyl-oleoyl-phosphatidylcholine, stearoyl-linoleoylphosphatidylcholine and palmitoyl-linoleoyl-phosphatidylcholine. Assymetric phosphatidylcholines are referred to as 1-acyl, 2-acyl-sn-glycero-3-phosphocholines, wherein the acyl groups are different from each other. Symmetric phosphatidylcholines are referred to as 1,2-diacyl-sn-glycero-3-phosphocholines. As used herein, the abbreviation “PC” refers to phosphatidylcholine. The phosphatidylcholine 1,2-dimyristoyl-sn-glycero-3-phosphocholine is abbreviated herein as “DMPC.” The phosphatidylcholine 1,2-dioleoyl-sn-glycero-3-phosphocholine is abbreviated herein as “DOPC.” The phosphatidylcholine 1,2-dipalmitoyl-sn-glycero-3-phosphocholine is abbreviated herein as “DPPC.”

In general, saturated acyl groups found in various lipids include groups having the trivial names propionyl, butanoyl, pentanoyl, caproyl, heptanoyl, capryloyl, nonanoyl, capryl, undecanoyl, lauroyl, tridecanoyl, myristoyl, pentadecanoyl, palmitoyl, phytanoyl, heptadecanoyl, stearoyl, nonadecanoyl, arachidoyl, heneicosanoyl, behenoyl, trucisanoyl and lignoceroyl. The corresponding IUPAC names for saturated acyl groups are trianoic, tetranoic, pentanoic, hexanoic, heptanoic, octanoic, nonanoic, decanoic, undecanoic, dodecanoic, tridecanoic, tetradecanoic, pentadecanoic, hexadecanoic, 3,7,11,15-tetramethylhexadecanoic, heptadecanoic, octadecanoic, nonadecanoic, eicosanoic, heneicosanoic, docosanoic, trocosanoic and tetracosanoic. Unsaturated acyl groups found in both symmetric and asymmetric phosphatidylcholines include myristoleoyl, palmitoleyl, oleoyl, elaidoyl, linoleoyl, linolenoyl, eicosenoyl and arachidonoyl. The corresponding IUPAC names for unsaturated acyl groups are 9-cis-tetradecanoic, 9-cis-hexadecanoic, 9-cis-octadecanoic, 9-trans-octadecanoic, 9-cis-12-cis-octadecadienoic, 9-cis-12-cis-15-cis-octadecatrienoic, 11-cis-eicosenoic and 5-cis-8-cis-11-cis-14-cis-eicosatetraenoic.

Exemplary phosphatidylethanolamines include dimyristoyl-phosphatidylethanolamine, dipalmitoyl-phosphatidylethanolamine, distearoyl-phosphatidylethanolamine, dioleoyl-phosphatidylethanolamine and egg phosphatidylethanolamine. Phosphatidylethanolamines may also be referred to under IUPAC naming systems as 1,2-diacyl-sn-glycero-3-phosphoethanolamines or 1-acyl-2-acyl-sn-glycero-3-phosphoethanolamine, depending on whether they are symmetric or assymetric lipids.

Exemplary phosphatidic acids include dimyristoyl phosphatidic acid, dipalmitoyl phosphatidic acid and dioleoyl phosphatidic acid. Phosphatidic acids may also be referred to under IUPAC naming systems as 1,2-diacyl-sn-glycero-3-phosphate or 1-acyl-2-acyl-sn-glycero-3-phosphate, depending on whether they are symmetric or assymetric lipids.

Exemplary phosphatidylserines include dimyristoyl phosphatidylserine, dipalmitoyl phosphatidylserine, dioleoylphosphatidylserine, distearoyl phosphatidylserine, palmitoyl-oleylphosphatidylserine and brain phosphatidylserine. Phosphatidylserines may also be referred to under IUPAC naming systems as 1,2-diacyl-sn-glycero-3-[phospho-L-serine] or 1-acyl-2-acyl-sn-glycero-3-[phospho-L-serine], depending on whether they are symmetric or assymetric lipids. As used herein, the abbreviation “PS” refers to phosphatidylserine.

Exemplary phosphatidylglycerols include dilauryloylphosphatidylglycerol, dipalmitoylphosphatidylglycerol, distearoylphosphatidylglycerol, dioleoyl-phosphatidylglycerol, dimyristoylphosphatidylglycerol, palmitoyl-oleoyl-phosphatidylglycerol and egg phosphatidylglycerol. Phosphatidylglycerols may also be referred to under IUPAC naming systems as 1,2-diacyl-sn-glycero-3-[phospho-rac-(1-glycerol)] or 1-acyl-2-acyl-sn-glycero-3-[phospho-rac-(1-glycerol)], depending on whether they are symmetric or assymetric lipids. The phosphatidylglycerol 1,2-dimyristoyl-sn-glycero-3-[phospho-rac-(1-glycerol)] is abbreviated herein as “DMPG”. The phosphatidylglycerol 1,2-dipalmitoyl-sn-glycero-3-(phospho-rac-1-glycerol) (sodium salt) is abbreviated herein as “DPPG”.

Suitable sphingomyelins might include brain sphingomyelin, egg sphingomyelin, dipalmitoyl sphingomyelin, and distearoyl sphingomyelin.

Other suitable lipids include glycolipids, sphingolipids, ether lipids, glycolipids such as the cerebrosides and gangliosides, and sterols, such as cholesterol or ergosterol. As used herein, the term cholesterol is sometimes abbreviated as “Chol.” Additional lipids suitable for use in liposomes are known to persons of skill in the art and are cited in a variety of sources, such as 1998 McCutcheon's Detergents and Emulsifiers, 1998 McCutcheon's Functional Materials, both published by McCutcheon Publishing Co., New Jersey, and the Avanti Polar Lipids, Inc. Catalog.

The overall surface charge of the liposome can affect the tissue uptake of a liposome. In certain embodiments of the present invention anionic phospholipids such as phosphatidylserine, phosphatidylinositol, phosphatidic acid, and cardiolipin are used. Neutral lipids such as dioleoylphosphatidyl ethanolamine (DOPE) may be used to target uptake of liposomes by specific tissues or to increase circulation times of intravenously administered liposomes. Cationic lipids may be used for alteration of liposomal charge, where the cationic lipid can be included as a minor component of the lipid composition or as a major or sole component. Suitable cationic lipids typically have a lipophilic moiety, such as a sterol, an acyl or diacyl chain, and where the lipid has an overall net positive charge. Preferably, the head group of the lipid carries the positive charge.

One of skill in the art will select vesicle-forming lipids that achieve a specified degree of fluidity or rigidity. The fluidity or rigidity of the liposome can be used to control factors such as the stability of the liposome in serum or the rate of release of the entrapped agent in the liposome. Liposomes having a more rigid lipid bilayer, or a liquid crystalline bilayer, are achieved by incorporation of a relatively rigid lipid. The rigidity of the lipid bilayer correlates with the phase transition temperature of the lipids present in the bilayer. Phase transition temperature is the temperature at which the lipid changes physical state and shifts from an ordered gel phase to a disordered liquid crystalline phase. Several factors affect the phase transition temperature of a lipid including hydrocarbon chain length and degree of unsaturation, charge and headgroup species of the lipid. Lipid having a relatively high phase transition temperature will produce a more rigid bilayer. Other lipid components, such as cholesterol, are also known to contribute to membrane rigidity in lipid bilayer structures. Cholesterol is widely used by those of skill in the art to manipulate the fluidity, elasticity and permeability of the lipid bilayer. It is thought to function by filling in gaps in the lipid bilayer. In contrast, lipid fluidity is achieved by incorporation of a relatively fluid lipid, typically one having a lower phase transition temperature. Phase transition temperatures of many lipids are tabulated in a variety of sources, such as Avanti Polar Lipids catalogue and Lipidat by Martin Caffrey, CRC Press.

Liposomes are preferably made from endogenous phospholipids such as dimyristoyl phosphatidylcholine (DMPC) and dimyristoyl phosphatidylglycerol (DMPG), phosphatidyl serine, phosphatidyl choline, dioleoyphosphatidyl choline [DOPC], cholesterol (CHOL) and cardiolipin.

1. Surface Modification

The use of saturated phospholipids and cholesterol in the formulation of liposome delivery systems cannot fully overcome their binding with serum components, and the consequently decreased MPS uptake of the vesicles: saturation of the MPS with a previous administration of “empty” liposomes may be necessary. Moreover, SUVs possess the disadvantage of low aqueous entrapment volume, and the use of charged liposomes can be toxic. These are overcome by coating the surface of the liposomes with inert molecules to faun a spatial barrier. By reducing MPS uptake, long-circulating liposomes can passively accumulate inside other tissues or organs. This phenomenon, called passive targeting, is especially evident in solid tumors undergoing angiogenesis: the presence of a discontinuous endothelial lining in the tumor vasculature during angiogenesis facilitates extravasation of liposomal formulations into the interstitial space, where they accumulate due to the lack of efficient lymphatic drainage of the tumor, and function as a sustained drug-release system. This causes the preferential accumulation of liposomes in the tumor area (a process known as enhanced permeation and retention effect or EPR). Liposome formulations do not extravasate from the bloodstream into normal tissues that have tight junctions between capillary endothelial cells. These mechanisms appear to be responsible for the improved therapeutic effects of liposomal anticancer drugs versus free drugs.

Among the different polymers investigated in attempts to improve the blood circulation time of liposomes, poly-(ethylene glycol) (PEG) has been widely used as polymeric steric stabilizer. It can be incorporated on the liposomal surface in different ways, but the most widely used method at present is to anchor the polymer in the liposomal membrane via a cross-linked lipid (ie, PEG-distearoylphosphatidylethanolamine [DSPE]. PEG (CAS number 25322-68-3) is a linear polyether diol with many useful properties, such as biocompatibility (Powell G M. Polyethylene glycol. In: Davidson R L, editor. Handbook of water soluble gums and resins. McGraw-Hill: 1980. pp. 18-31), solubility in aqueous and organic media, lack of toxicity, very low immunogenicity and antigenicity (Dreborg et al. Crit Rev Ther Drug Carrier Syst. 1990: 315-65), and good excretion kinetics (Yamaoka et al. J Pharm Sci. 1994; 83:601-6). The molecular weight and structure of PEG molecules can be freely modulated for specific purposes, and it is easier and cheaper to conjugate the polymer with the lipid.

Poly-ethylene glycols have been used to derivatize therapeutic proteins and peptides, increasing drug stability and solubility, lowering toxicity, increasing half-life (Caliceti et al. Adv Drug Del Rev. 2003; 55:1261-77), decreasing clearance and immunogenicity. These benefits have been particularly observed using branched PEG in the derivatization (Monfardini et al. Bioconj Chem. 1998; 9:418-50). For the most part, reaction with PEG derivatives does not alter the mechanism of action of a therapeutic protein; rather it enhances its therapeutic effect by altering its pharmacokinetics. PEG-ademase (utilized to treat immunodeficiency), PEG-visomant (human growth hormone), PEG-aspargase (for leukemias), PEG-interferon-alpha (for chronic hepatitis C), PEG-aldesleukin (PEG-IL-2) (an anticancer agent), and PEG-filgrastim (for chemotherapy-induced transferase neutropenia) are the principal PEGylated proteins in clinical use (Mahmood et al. Clin Pharmacokinet. 2005; 44:331-47).

Surface modification of liposomes with PEG can be achieved in several ways: by physically adsorbing the polymer onto the surface of the vesicles, by incorporating the PEG-lipid conjugate during liposome preparation, or by covalently attaching reactive groups onto the surface of preformed liposomes. Grafting PEG onto liposomes has demonstrated several biological and technological advantages. The most significant properties of PEGylated vesicles are their strongly reduced MPS uptake and their prolonged blood circulation and thus improved distribution in perfused tissues. Moreover, the PEG chains on the liposome surface avoid vesicle aggregation, improving stability of formulations.

The behavior of PEGylated liposomes depends on the characteristics and properties of the specific PEG linked to the surface. The molecular mass of the polymer, as well as the graft density, determine the degree of surface coverage and the distance between graft sites. The most evident characteristic of PEG-grafted liposomes (PEGylated-liposomes) is their circulation longevity, regardless of surface charge or the inclusion of stabilizing agent such as cholesterol. In liposomes composed of phospholipids and cholesterol, the ability of PEG to increase the circulation lifetime of the vehicles has been found to depend on both the amount of grafted PEG and the length or molecular weight of the polymer (Allen et al. Biochim Biophys Acta. 1991; 1066:29-36.

Liposomes, coated with one or more materials that promote diffusion of the particles through mucosa are disclosed. Examples of the surface-altering agents include, but are not limited to, polyethylene glycol (“PEG”) and poloxomers (polyethylene oxide block copolymers). Poly(ethylene glycol) (PEG) are macromolecules which can be used for modification of biological macromolecules and many pharmaceutical and biotechnological applications. Liposomes can be modified by combining them with PEG.

i. Polyethylene Glycol (PEG)

A preferred coating agent is poly(ethylene glycol), also known as PEG. PEG may be employed to reduce adhesion in brain ECM in certain configurations, e.g., wherein the length of PEG chains extending from the surface is controlled (such that long, unbranched chains that interpenetrate into the ECM are reduced or eliminated). For example, linear high MW PEG may be employed in the preparation of particles such that only portions of the linear strands extend from the surface of the particles (e.g., portions equivalent in length to lower MW PEG molecules). Alternatively, branched high MW PEG may be employed. In such embodiments, although the molecular weight of a PEG molecule may be high, the linear length of any individual strand of the molecule that extends from the surface of a particle would correspond to a linear chain of a lower MW PEG molecule.

Representative PEG molecular weights in daltons (Da) include 300 Da, 600 Da, 1 kDa, 2 kDa, 3 kDa, 4 kDa, 5 kDa, 6 kDa, 8 kDa, 10 kDa, 15 kDa, 20 kDa, 30 kDa, 50 kDa, 100 kDa, 200 kDa, 500 kDa, and 1 MDa. In preferred embodiments, the PEG has a molecular weight of about 2,000 to 5,000 Daltons. PEG of any given molecular weight may vary in other characteristics such as length, density, and branching. In a particular embodiment, a coating agent is methoxy-PEG-amine, with a MW of 5 kDa. In another embodiment, a coating agent is methoxy-PEG-N-hydroxysuccinimide with a MW of 5 kDa (mPEG-NHS 5 kDa).

In alternative embodiments, the coating is a poloxamer such as the polyethylene glycol-polyethylene oxide block copolymers marketed as PLUORONICs®.

PEG alternative polymers should be soluble, hydrophilic, have highly flexible main chain, and high biocompatibility. Synthetic polymers, such as poly(vinyl pyrrolidone) (PVP) and poly(acryl amide) (PAA), are the most prominent examples of other potentially protective polymers (Torchilin et al Biochim Biophys Acta. 1994 Oct. 12; 1195(1):181-4; Biochim Biophys Acta. 1994 Oct. 12; 1195(1):11-20; J Pharm Sci. 1995 September; 84(9):1049-53). Liposomes containing DSPE covalently linked to poly(2-methyl-2-oxazoline) or to poly(2-ethyl-2-oxazoline) also exhibit extended blood circulation time and decreased uptake by the liver and spleen (Woodle, et al. Bioconjug Chem. 1994 November-December; 5(6):493-6). Similar observations have been reported for phosphatidyl polyglycerols (Unezaki, et al. Pharm Res. 1994 August; 11(8):1180-5).

More recent papers describe long circulating liposomes prepared using poly[N-(2-hydroxypropyl) methacrylamide] (Whiteman, et al. J Liposome Res. 2001; 11(2-3):153-64), amphiphilic poly-N-vinylpyrrolidones (Torchilin Biomaterials. 2001 November; 22(22):3035-44.), L-amino-acid-based biodegradable polymer-lipid conjugates (Metselaar, et al. Bioconjug Chem. 2003 November-December; 14(6):1156-64), and polyvinyl alcohol (Takeuchi, et al. Eur. J. Pharm. Biopharm, 2012 February; 80(2):340-6. doi:10.1016/j.ejpb.2011.10.011. Epub 2011 Oct. 20.). All groups of polymer-coated liposomes reported have been found to extend blood circulation time, while liver capture was diminished. These results are comparable with those for PEG-liposomes; the efficacy of the steric effect quite naturally depends on the quantity of incorporated polymer. The prolonged circulation time of polyvinyl alcohol-(molecular weight: 20000) coated liposomes (1.3 mol % coating) was comparable with that of a stealth liposome prepared with 8 mol % of DSPE-PEG2000.

Also, L-amino-acid-based polymers also showed prolonged circulation time and reduced uptake by the MPS, to the same extent as DSPE-PEG2000. Furthermore, these polymers appear to be attractive alternatives for designing long-circulation liposomes, because they have the advantage of being biodegradable.

PEG-coated liposomes have also been shown to increase mucosal penetration. See, for example, Li, et al. Int. J. Nanomed. 2011: 6,3151-3162 and WO2013166498 by The Johns Hopkins University. It should be noted that the formulations described herein represent a subset with improved mucosal penetration as compared to PEG-coated liposomes generally, as demonstrated by the examples, showing that there is a narrow range of the ratio of PEG-lipid to lipid mol % to provide optimized mucosal penetration.

ii. Density of Coating Agent

In preferred embodiments the liposomes are coated with PEG or other coating agents at a density that optimizes rapid diffusion through the brain parenchyma. The density of the coating can be varied based on a variety of factors including the material and the composition of the particle.

For liposomes, the composition is usually defined by the molar ratio between PEG-lipid and non-PEGylated-lipid. These can range from three to elevent mol %. Most preferably the ratio of PEG-lipid to non-PEGylated-lipid is about 7 mol %.

2. Liposome Formation and Drug Entrapment

The formation and use of liposomes is generally known to those of skill in the art, as described in, e.g. Liposome Technology, Vols. 1, 2 and 3, Gregory Gregoriadis, ed., CRC Press, Inc; Liposomes: Rational Design, Andrew S. Janoff, ed., Marcel Dekker, Inc.; Medical Applications of Liposomes, D. D. Lasic and D. Papahadjopoulos, eds., Elsevier Press; Bioconjugate Techniques, by Greg T. Hermanson, Academic Press; and Pharmaceutical Manufacturing of Liposomes, by Francis J. Martin, in Specialized Drug Delivery Systems (Praveen Tyle, Ed.), Marcel Dekker, Inc.

The original method of forming liposomes (Bangham et al., 1965, J. Mol. Biol. 13: 238-252) involved first suspending phospholipids in an organic solvent and then evaporating to dryness until a dry lipid cake or film is formed. An appropriate amount of aqueous medium is added and the lipids spontaneously form multilamellar concentric bilayer vesicles (also termed multilamellar vesicles (MLVs). These MLVs can then be dispersed by mechanical means. MLVs generally have diameters of from 25 nm to 4 .mu.m. Sonication of MLVs results in the formation of small unilamellar vesicles (SUVs) with diameters in the range of 200 to 500 .ANG., containing an aqueous solution in the core. SUVs are smaller than MLVs and unilamellar.

While the original MLVs and SUVs were created using phospholipids, any of the lipid compositions described previously can be used to create MLVs and SUVs. When mixtures of lipids are used the lipids are typically co-dissolved in an organic solvent prior to the evaporation step of the process described above.

An alternate method of creating large unilamellar vesicles (LUVs) is the reverse-phase evaporation process, described, for example, in U.S. Pat. No. 4,235,871. This process generates reverse-phase evaporation vesicles (REVs), which are mostly unilamellar but also typically contain some oligolamellar vesicles. In this procedure a mixture of polar lipid in an organic solvent is mixed with a suitable aqueous medium. A homogeneous water-in-oil type of emulsion is formed and the organic solvent is evaporated until a gel is formed. The gel is then converted to a suspension by dispersing the gel-like mixture in an aqueous media.

Liposomes may also be prepared wherein the liposomes have substantially homogeneous sizes in a selected size range. One effective sizing method for REVs and MLVs involves extruding an aqueous suspension of the liposomes through a series of polycarbonate membranes having a selected uniform pore size in the range of 0.03 to 0.2 micron, typically 0.05, 0.08, 0.1, or 0.2 microns. The pore size of the membrane corresponds roughly to the largest sizes of liposomes produced by extrusion through that membrane, particularly where the preparation is extruded two or more times through the same membrane. Homogenization methods are also useful for down-sizing liposomes to sizes of 100 nm or less (Martin, F. J., in Specialized Drug Delivery Systems-Manufacturing and Production Technology, (P. Tyle, Ed.) Marcel Dekker, New York, pp. 267-316 (1990)). Homogenization relies on shearing energy to fragment large liposomes into smaller ones. Other appropriate methods of down-sizing liposomes include reducing liposome size by vigorous agitation of the liposomes in the presence of an appropriate solubilizing detergent, such as deoxycholate.

B. Therapeutic, Prophylactic, and Diagnostic Agents to be Delivered

1. Therapeutic and Prophylactic Agents

In some embodiments, the particles have encapsulated therein, dispersed therein, and/or covalently or non-covalently associate with the surface one or more therapeutic agents. The therapeutic agent can be a small molecule, protein, polysaccharide or saccharide, nucleic acid molecule and/or lipid.

Any protein can be formulated, including recombinant, isolated, or synthetic proteins, glycoproteins, or lipoproteins. These may be antibodies (including antibody fragments and recombinant antibodies), enzymes, growth factors or homones, immunomodifiers, antiinfectives, antiproliferatives, or other therapeutic, prophylactic, or diagnostic proteins. In certain embodiments, the protein has a molecular weight greater than about 150 kDa, greater than 160 kDa, greater than 170 kDa, greater than 180 kDa, greater than 190 kDa or even greater than 200 kDa. In certain embodiments, the protein can be a PEGylated protein.

Exemplary classes of small molecule therapeutic agents include, but are not limited to, analgesics, anti-inflammatory drugs, antipyretics, antidepressants, antiepileptics, antiopsychotic agents, neuroprotective agents, anti-proliferatives, such as anti-cancer agent, anti-infectious agents, such as antibacterial agents and antifungal agents, antihistamines, antimigraine drugs, antimuscarinics, anxioltyics, sedatives, hypnotics, antipsychotics, bronchodilators, anti-asthma drugs, cardiovascular drugs, corticosteroids, dopaminergics, electrolytes, gastro-intestinal drugs, muscle relaxants, nutritional agents, vitamins, parasympathomimetics, stimulants, anorectics and anti-narcoleptics.

In some embodiments, the agent is one or more nucleic acids. The nucleic acid can alter, correct, or replace an endogenous nucleic acid sequence. The nucleic acid can be used to treat cancers, correct defects in genes in pulmonary diseases and metabolic diseases affecting lung function, for example, to treat of Parkinsons and ALS where the genes reach the brain through nasal delivery.

Gene therapy is a technique for correcting defective genes responsible for disease development. Researchers may use one of several approaches for correcting faulty genes:

A normal gene may be inserted into a nonspecific location within the genome to replace a nonfunctional gene. This approach is most common.

An abnormal gene could be swapped for a normal gene through homologous recombination.

The abnormal gene could be repaired through selective reverse mutation, which returns the gene to its normal function.

The regulation (the degree to which a gene is turned on or off) of a particular gene could be altered.

The nucleic acid can be a DNA, RNA, a chemically modified nucleic acid, or combinations thereof. For example, methods for increasing stability of nucleic acid half-life and resistance to enzymatic cleavage are known in the art, and can include one or more modifications or substitutions to the nucleobases, sugars, or linkages of the polynucleotide. The nucleic acid can be custom synthesized to contain properties that are tailored to fit a desired use. Common modifications include, but are not limited to use of locked nucleic acids (LNAs), unlocked nucleic acids (UNAs), morpholinos, peptide nucleic acids (PNA), phosphorothioate linkages, phosphonoacetate linkages, propyne analogs, 2′-O-methyl RNA, 5-Me-dC, 2′-5′ linked phosphodiester linage, Chimeric Linkages (Mixed phosphorothioate and phosphodiester linkages and modifications), conjugation with lipid and peptides, and combinations thereof.

2. Diagnostic Agents

Exemplary diagnostic materials include paramagnetic molecules, fluorescent compounds, magnetic molecules, and radionuclides. Suitable diagnostic agents include, but are not limited to, x-ray imaging agents and contrast media. Radionuclides also can be used as imaging agents. Examples of other suitable contrast agents include gases or gas emitting compounds, which are radioopaque. Liposomes can further include agents useful for determining the location of administered particles. Agents useful for this purpose include fluorescent tags, radionuclides and contrast agents.

For those embodiments where the one or more therapeutic, prophylactic, and/or diagnostic agents are encapsulated within a polymeric liposome and/or associated with the surface of the liposome, the percent drug loading is from about 1% to about 80%, from about 1% to about 50%, from about 1% to about 40% by weight, from about 1% to about 20% by weight, or from about 1% to about 10% by weight. Amounts vary based on the lipid and compound to be encapsulated, and the conditions used to form the encapsulating liposomes. The ranges above are inclusive of all values from 1% to 80%. For those embodiments where the agent is associated with the surface of the particle, the percent loading may be higher since the amount of drug is not limited by the methods of encapsulation. In some embodiments, the agent to be delivered may be encapsulated within a liposome and associated with the surface of the particle. Nutraceuticals can also be incorporated. These may be vitamins, supplements such as calcium or biotin, or natural ingredients such as plant extracts or phytohormones.

In a preferred embodiment, liposomes are formed by the lipid film hydration method. In brief, lipid mixture (for example, DSPC:Cholesterol at a molar ratio of 63%:37%, with addition of different amount of DSPE-PEG₂ₖ) dissolved in a solvent such as chloroform is dried, and the resultant thin film hydrated using deionized water (D₂O) with 1% w/w DSS to form multilamellar vesicles. The mixture is then annealed at 65-70° C. for one hour, sonicated, and subsequently extruded through stacked polycarbonate filters (pore size 400 nm and then 100 nm).

### III. Methods of Use

A. Pharmaceutical Preparations

The formulations described herein contain an effective amount of liposomes in a pharmaceutical carrier appropriate for administration to a mucosal surface. The formulations can be administered parenterally (e.g., by injection or infusion), topically (e.g., to the eye, vaginally, rectally, or orally), or via pulmonary administration.

1. Pulmonary Formulations

Pharmaceutical formulations and methods for the pulmonary administration of active agents to patients are known in the art.

The respiratory tract is the structure involved in the exchange of gases between the atmosphere and the blood stream. The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchioli which then lead to the ultimate respiratory zone, the alveoli, or deep lung, where the exchange of gases occurs.

Formulations can be divided into dry powder formulations and liquid formulations. Both dry powder and liquid formulations can be used to form aerosol formulations. The term aerosol as used herein refers to any preparation of a fine mist of particles, which can be in solution or a suspension, whether or not it is produced using a propellant.

Dry powder formulations are finely divided solid formulations containing liposome carriers which are suitable for pulmonary administration. Dry powder formulations include, at a minimum, one or more liposome carriers which are suitable for pulmonary administration. Such dry powder formulations can be administered via pulmonary inhalation to a patient without the benefit of any carrier, other than air or a suitable propellant.

In other embodiments, the dry powder formulations contain one or more liposome gene carriers in combination with a pharmaceutically acceptable carrier. In these embodiments, the liposome gene carriers and pharmaceutical carrier can be formed into nano- or microparticles for delivery to the lung.

The pharmaceutical carrier may include a bulking agent or a lipid or surfactant. Natural surfactants such as dipalmitoylphosphatidylcholine (DPPC) are the most preferred. Synthetic and animal derived pulmonary surfactants include:

Synthetic Pulmonary Surfactants
Exosurf—a mixture of DPPC with hexadecanol and tyloxapol added as spreading agents
Pumactant (Artificial Lung Expanding Compound or ALEC)—a mixture of DPPC and PG
KL-4—composed of DPPC, palmitoyl-oleoyl phosphatidylglycerol, and palmitic acid, combined with a 21 amino acid synthetic peptide that mimics the structural characteristics of SP-B.
Venticute—DPPC, PG, palmitic acid and recombinant SP-C

Animal Derived Surfactants
Alveofact—extracted from cow lung lavage fluid
Curosurf—extracted from material derived from minced pig lung
Infasurf—extracted from calf lung lavage fluid
Survanta—extracted from minced cow lung with additional DPPC, palmitic acid and tripalmitin

Exosurf, Curosurf, Infasurf, and Survanta are the surfactants currently FDA approved for use in the U.S.

The pharmaceutical carrier may also include one or more stabilizing agents or dispersing agents. The pharmaceutical carrier may also include one or more pH adjusters or buffers. Suitable buffers include organic salts prepared from organic acids and bases, such as sodium citrate or sodium ascorbate. The pharmaceutical carrier may also include one or more salts, such as sodium chloride or potassium chloride.

Dry powder formulations are typically prepared by blending one or more liposome carriers with one or more pharmaceutically acceptable carriers. Optionally, additional active agents may be incorporated into the mixture as discussed below. The mixture is then formed into particles suitable for pulmonary administration using techniques known in the art, such as lyophilization, spray drying, agglomeration, spray coating, coacervation, low temperature casting, milling (e.g., air-attrition milling (jet milling), ball milling), high pressure homogenization, and/or supercritical fluid crystallization.

An appropriate method of particle formation can be selected based on the desired particle size, particle size distribution, and particle morphology desired for the formulation. In some cases, the method of particle formation is selected so as to produce a population of particles with the desired particle size, particle size distribution for pulmonary administration. Alternatively, the method of particle formation can produce a population of particles from which a population of particles with the desired particle size, particle size distribution for pulmonary administration is isolated, for example by sieving.

Dry powder formulations can be administered as dry powder using suitable methods known in the art. Alternatively, the dry powder formulations can be suspended in the liquid formulation s described below, and administered to the lung using methods known in the art for the delivery of liquid formulations.

Liquid formulations contain one or more liposome carriers suspended in a liquid pharmaceutical carrier.

Suitable liquid carriers include, but are not limited to distilled water, de-ionized water, pure or ultrapure water, saline, and other physiologically acceptable aqueous solutions containing salts and/or buffers, such as phosphate buffered saline (PBS), Ringer's solution, and isotonic sodium chloride, or any other aqueous solution acceptable for administration to an animal or human.

Preferably, liquid formulations are isotonic relative to physiological fluids and of approximately the same pH, ranging e.g., from about pH 4.0 to about pH 7.4, more preferably from about pH 6.0 to pH 7.0. The liquid pharmaceutical carrier can include one or more physiologically compatible buffers, such as a phosphate buffers. One skilled in the art can readily determine a suitable saline content and pH for an aqueous solution for pulmonary administration.

Liquid formulations may include one or more suspending agents, such as cellulose derivatives, sodium alginate, polyvinylpyrrolidone, gum tragacanth, or lecithin. Liquid formulations may also include one or more preservatives, such as ethyl or n-propyl p-hydroxybenzoate.

In some cases the liquid formulation may contain one or more solvents that are low toxicity organic (i.e. nonaqueous) class 3 residual solvents, such as ethanol, acetone, ethyl acetate, tetrahydofuran, ethyl ether, and propanol. These solvents can be selected based on their ability to readily aerosolize the formulation. Any such solvent included in the liquid formulation should not detrimentally react with the one or more active agents present in the liquid formulation. The solvent should be sufficiently volatile to enable formation of an aerosol of the solution or suspension. Additional solvents or aerosolizing agents, such as a freon, alcohol, glycol, polyglycol, or fatty acid, can also be included in the liquid formulation as desired to increase the volatility and/or alter the aerosolizing behavior of the solution or suspension.

Liquid formulations may also contain minor amounts of polymers, surfactants, or other excipients well known to those of the art. In this context, “minor amounts” means no excipients are present that might adversely affect uptake of the one or more active agents in the lungs.

The dry powder and liquid formulations described above can be used to form aerosol formulations for pulmonary administration. Aerosols for the delivery of therapeutic agents to the respiratory tract are known in the art. The term aerosol as used herein refers to any preparation of a fine mist of solid or liquid particles suspended in a gas. In some cases, the gas may be a propellant; however, this is not required. Aerosols may be produced using a number of standard techniques, including as ultrasonication or high pressure treatment.

In some cases, a device is used to administer the formulations to the lungs. Suitable devices include, but are not limited to, dry powder inhalers, pressurized metered dose inhalers, nebulizers, and electrohydrodynamic aerosol devices.

Inhalation can occur through the nose and/or the mouth of the patient. Administration can occur by self-administration of the formulation while inhaling or by administration of the formulation via a respirator to a patient on a respirator.

2. Parenteral and Enteral Formulations

In some embodiments, the liposomes are formulated for parenteral delivery, such as injection or infusion, in the faun of a solution or suspension. The formulation can be administered via any route, such as, the blood stream or directly to the organ or tissue to be treated. In some embodiments, the liposomes are formulated for parenteral formulation to the eye.

“Parenteral administration”, as used herein, means administration by any method other than through the digestive tract or non-invasive topical or regional routes. For example, parenteral administration may include administration to a patient intravenously, intradermally, intraperitoneally, intrapleurally, intratracheally, intramuscularly, subcutaneously, subjunctivally, by injection, and by infusion.

Parenteral formulations can be prepared as aqueous compositions using techniques is known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or phatmacologically acceptable salts thereof can be prepared in water or another solvent or dispersing medium suitably mixed with one or more pharmaceutically acceptable excipients including, but not limited to, surfactants, dispersants, emulsifiers, pH modifying agents, and combination thereof.

Suitable surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-beta-alanine, sodium N-lauryl-beta-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Water soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

3. Ocular Formulations

Pharmaceutical formulations for ocular administration are preferably in the form of a sterile aqueous solution or suspension of particles formed from one or more polymer-drug conjugates. Acceptable solvents include, for example, water, Ringer's solution, phosphate buffered saline (PBS), and isotonic sodium chloride solution. The formulation may also be a sterile solution, suspension, or emulsion in a nontoxic, parenterally acceptable diluent or solvent such as 1,3-butanediol.

In some instances, the formulation is distributed or packaged in a liquid form. Alternatively, formulations for ocular administration can be packed as a solid, obtained, for example by lyophilization of a suitable liquid formulation. The solid can be reconstituted with an appropriate carrier or diluent prior to administration.

Solutions, suspensions, or emulsions for ocular administration may be buffered with an effective amount of buffer necessary to maintain a pH suitable for ocular administration. Suitable buffers are well known by those skilled in the art and some examples of useful buffers are acetate, borate, carbonate, citrate, and phosphate buffers.

Solutions, suspensions, or emulsions for ocular administration may also contain one or more tonicity agents to adjust the isotonic range of the formulation. Suitable tonicity agents are well known in the art and some examples include glycerin, mannitol, sorbitol, sodium chloride, and other electrolytes.

Solutions, suspensions, or emulsions for ocular administration may also contain one or more preservatives to prevent bacterial contamination of the ophthalmic preparations. Suitable preservatives are known in the art, and include polyhexamethylenebiguanidine (PHMB), benzalkonium chloride (BAK), stabilized oxychloro complexes (otherwise known as Purite®), phenylmercuric acetate, chlorobutanol, sorbic acid, chlorhexidine, benzyl alcohol, parabens, thimerosal, and mixtures thereof.

Solutions, suspensions, or emulsions for ocular administration may also contain one or more excipients known art, such as dispersing agents, wetting agents, and suspending agents.

4. Topical Formulations

In still other embodiments, the liposomes are formulated for topical administration to mucosa. Suitable dosage forms for topical administration include creams, ointments, salves, sprays, gels, lotions, emulsions, liquids, and transdermal patches. The formulation may be formulated for transmucosal, transepithelial, transendothelial, or transdermal administration. The compositions contain one or more chemical penetration enhancers, membrane permeability agents, membrane transport agents, emollients, surfactants, stabilizers, and combination thereof.

In some embodiments, the liposomes can be administered as a liquid formulation, such as a solution or suspension, a semi-solid formulation, such as an lotion or ointment, or a solid formulation. In some embodiments, the liposomes are formulated as liquids, including solutions and suspensions, such as eye drops or as a semi-solid formulation, such as ointment or lotion for topical application to mucosa, such as the eye or vaginally or rectally.

The formulation may contain one or more excipients, such as emollients, surfactants, emulsifiers, and penetration enhancers.

“Emollients” are an externally applied agent that softens or soothes skin and are generally known in the art and listed in compendia, such as the “Handbook of Pharmaceutical Excipients”, 4^{th} Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

Suitable classes of penetration enhancers are known in the art and include, but are not limited to, fatty alcohols, fatty acid esters, fatty acids, fatty alcohol ethers, amino acids, phospholipids, lecithins, cholate salts, enzymes, amines and amides, complexing agents (liposomes, cyclodextrins, modified celluloses, and diimides), macrocyclics, such as macrocylic lactones, ketones, and anhydrides and cyclic ureas, surfactants, N-methyl pyrrolidones and derivatives thereof, DMSO and related compounds, ionic compounds, azone and related compounds, and solvents, such as alcohols, ketones, amides, polyols (e.g., glycols). Examples of these classes are known in the art.

“Hydrophilic” as used herein refers to substances that have strongly polar groups that readily interact with water.

“Lipophilic” refers to compounds having an affinity for lipids.

“Amphiphilic” refers to a molecule combining hydrophilic and lipophilic (hydrophobic) properties

“Hydrophobic” as used herein refers to substances that lack an affinity for water; tending to repel and not absorb water as well as not dissolve in or mix with water.

A “gel” is a colloid in which the dispersed phase has combined with the continuous phase to produce a semisolid material, such as jelly.

An “oil” is a composition containing at least 95% wt of a lipophilic substance. Examples of lipophilic substances include but are not limited to naturally occurring and synthetic oils, fats, fatty acids, lecithins, triglycerides and combinations thereof.

A “continuous phase” refers to the liquid in which solids are suspended or droplets of another liquid are dispersed, and is sometimes called the external phase. This also refers to the fluid phase of a colloid within which solid or fluid particles are distributed. If the continuous phase is water (or another hydrophilic solvent), water-soluble or hydrophilic drugs will dissolve in the continuous phase (as opposed to being dispersed). In a multiphase formulation (e.g., an emulsion), the discreet phase is suspended or dispersed in the continuous phase.

An “emulsion” is a composition containing a mixture of non-miscible components homogenously blended together. In particular embodiments, the non-miscible components include a lipophilic component and an aqueous component. An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A sub-set of emulsions are the self-emulsifying systems. These drug delivery systems are typically capsules (hard shell or soft shell) comprised of the drug dispersed or dissolved in a mixture of surfactant(s) and lipophilic liquids such as oils or other water immiscible liquids. When the capsule is exposed to an aqueous environment and the outer gelatin shell dissolves, contact between the aqueous medium and the capsule contents instantly generates very small emulsion droplets. These typically are in the size range of micelles or liposomes. No mixing force is required to generate the emulsion as is typically the case in emulsion formulation processes.

A “lotion” is a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

A “cream” is a viscous liquid or semi-solid emulsion of either the “oil-in-water” or “water-in-oil type”. Creams may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments as they are generally easier to spread and easier to remove.

The difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

An “ointment” is a semisolid preparation containing an ointment base and optionally one or more active agents. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A “gel” is a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components. Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C₁₂-C₁₅ alkyl benzoates, mineral oil, squalane, cyclomethicone, capric/caprylic triglycerides, and combinations thereof.

Foams consist of an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Buffers are used to control pH of a composition. Preferably, the buffers buffer the composition from a pH of about 4 to a pH of about 7.5, more preferably from a pH of about 4 to a pH of about 7, and most preferably from a pH of about 5 to a pH of about 7. In a preferred embodiment, the buffer is triethanolamine.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

In certain embodiments, it may be desirable to provide continuous delivery of one or more noscapine analogs to a patient in need thereof. For topical applications, repeated application can be done or a patch can be used to provide continuous administration of the noscapine analogs over an extended period of time.

B. Methods of Administration

Liposomes can be administered enterally, topically, via the pulmonary, nasal, rectal, vaginal, or oral routes, to lumens, vessels or tissues having a mucosal coating therein. The formulations are administered to produce a therapeutic, prophylactic or diagnostic result.

The present invention will be further understood by reference to the following non-limiting examples.
Abbreviations: BA, barbituric acid; CVM, cervicovaginal mucus; diaCEST, diamagnetic chemical exchange saturation transfer; MPP, mucus-penetrating particles; MPT, multiple particle tracking; MRI, magnetic resonance imaging; PEG, polyethylene glycol.

### EXAMPLE 1

### Effect of PEG Surface Density on Liposome Mobility in Mucus

The composition of PEG-conjugated lipids was varied to investigate the effect of PEG surface density on liposome mobility in human cervicovaginal mucus (CVM) and vaginal distribution in vivo. The liposomal MPP were loaded with barbituric acid (BA), a water-soluble diamagnetic Chemical Exchange Saturation Transfer (diaCEST) contrast agent, and monitored the vaginal distribution and retention of the liposomes via Magnetic Resonance Imaging (MRI).

Methods and Materials

Liposomes composed of 1,2-disteoroyl-sn-glycero-3-phosphatidylcholine (DSPC), cholesterol, and 1,2-distearoyl-sn-glycerophosphoethanolamine poly(ethylene glycol)₂₀₀₀ (DSPE-PEG₂ₖ) were prepared and characterized following procedures adapted from previous reports. Ensign et al Sci Transl Med 2012; 4:138ra79; Chan et al J Control Release 2014; 180:51-9; Xu, et al. J Control Release 2013; 170:279-86. Data represent mean±standard error of the mean (S.E.M.).

Liposome Preparation and Basic Characterization

1,2-disteoroyl-sn-glycero-3-phosphatidylcholine (DSPC), and 1,2-distearoyl-sn-glycerophosphoethanolamine poly(ethylene glycol)₂₀₀₀ (DSPE-PEG₂ₖ) were obtained from Avanti Polar Lipids, Inc. (Alabaster, Ala.). Cholesterol, deuterium oxide (D₂O, containing 1% w/w 3-(trimethyl-silyl)-1-propanesulfonic acid sodium salt, or DSS) and barbituric acid (BA) were purchased from Sigma-Aldrich (St. Louis, Mo.). Liposomes were foamed by the lipid film hydration method. In brief, 25 mg of lipid mixture (DSPC:Cholesterol at a molar ratio of 63%:37%, with addition of different amount of DSPE-PEG₂ₖ) dissolved in chloroform was dried, and the resultant thin film was hydrated using 1 mL D₂O with 1% w/w DSS to form multilamellar vesicles. The mixture was then annealed at 65-70° C. for one hour, sonicated, and subsequently extruded through stacked polycarbonate filters (pore size 400 nm and then 100 nm). For in vivo distribution and imaging studies, BA-loaded liposomes were prepared following a similar procedure, in which the lipid mixture contained 1 mol % rhodamine-labeled 18:1 PE and the lipid thin film was hydraded with BA aqueous solution at 20 mg/mL. Freshly prepared liposomes were then filtered through SEPHADEX® G-50 gel columns (GE Healthcare Life Sciences, Pittsburgh, Pa.) to remove unloaded compounds, and stored at 4° C. prior to use. The size (number mean) and heterogeneity in size (polydispersity index, PDI) were measured in PBS at room temperature by dynamic light scattering (DLS) using a Nanosizer ZS90 (Malvern Instruments, Southborough, Mass.).

Characterization of Surface PEG Density of Liposomes

The actual molar ratio of DSPE-PEG₂ₖ in liposomes was determined. First, the 1H NMR spectrum of liposomes (prepared in D₂O, with 1% w/w DSS as internal reference) was measured using VARIAN INOVA® 500 instrument (Varian Inc., Palo Alto, Calif.) at 500 MHz, with relaxation time set at 10 s and ZG pulse at 90°.⁵ The amount of DSPE-PEG₂ₖ was then calculated based on the ratio between the intergrals of PEG peaks (3.3-4.1 ppm) vs. DSS reference peaks (−0.3-0.3 ppm), and a calibration curve prepared using standard samples of DSPE-PEG₂ₖ. Three hundred microliters of liposomes were then freeze-dried and weighed, and the net mass of lipids was calculated by subtracting the weight of 300 μL D₂O-1% DSS freeze-dried from the dried weight of the liposomes. The molar percentage of DSPE-PEG₂ₖ was then calculated using the following formula:

$$mol \% DSPE - {PEG}_{2 k} = \frac{\frac{{m}_{DSPE - PEG 2 k}}{{M}_{DSPE - PEG 2 k}}}{\frac{{m}_{DSPE - PEG 2 k}}{{M}_{DSPE - PEG 2 k}} + \frac{{m}_{total lipid} - {m}_{DSPE - PEG 2 k}}{{M}_{DSPC - Cholesterol}}} \times 100 \%$$

where M_{DSPE-PEG2k}=2802 g/mol and M_{DSPC-Cholesterol}=646 g/mol (weighted average MW based on a DSPC:Cholesterol ratio of 63%:37%), and m_{DSPE-PEG2k} and m_{total lipid} were detennined by the freeze-drying as described above.

The liposomal surface density of PEG was then estimated. The total surface area of a liposome (SA, including both inner and outer surfaces of the lipid bilayer), and the total number of lipid molecules in the lipid bilayer of a liposome (Nₜₒₜ), has the following relationship:

$${N}_{tot} = \frac{SA}{{a}_{ave}}$$

where aₐᵥₑ is the weighted average molecular surface area of the lipids. The following formula was used to estimate aₐᵥₑ:

aₐᵥₑ=w_{phospholipid}×a_{phospholipid}+w_{cholesterol}×a_{cholesterol}

where w_{phospholipid}=63%, w_{cholsterol}=37%, and a_{phospholipid}=0.55 nm² (with the condensation effect by cholesterol), a_{cholesterol}=0.27 nm². The resulting aₐᵥₑ=0.45 nm², which is close to estimates previously used (Suk, et al. J Control Release 2014; 178:8-17; Torchilin Nat Rev Drug Discov 2005; 4:145-60). While aₐᵥₑ could be slightly different at various PEGylation levels, constant value was assumed to maintain consistency for the subsequent calculations.

The liposomal surface density of PEG was then estimated using the following formula, assuming DSPE-PEG₂ₖ are uniformly distributed on both sides of the lipid bilayer:

$$\begin{matrix}PEG surface density = \frac{{N}_{tot} \times mol \% DSPE - PEG 2 k}{SA} \\ = \frac{mol \% DSPE - PEG 2 k}{{a}_{ave}}\end{matrix}$$

The conformation of PEG chains on the liposomal surface was evaluated. For each liposome, the full surface mushroom coverage [Γ], i.e., the surface area covered by all PEG molecules assuming they are in an unconstrained, mushroom conformation, is defined as:

[Γ]=PEG surface density×SA×πξ²

where ξ is the diameter of a theorectical spherical area occupied by a single, unconstrained PEG chain, estimated based on random-walk statistics as previously reported:⁹

ξ=0.76×M_{PEG}^{0.5} [Å]

Provided that M_{PEG}=2000 Da, the occupied area πξ² was estimated ˜9.1 nm². The ratio of [Γ] to the total surface area of a liposome, i.e., [Γ/SA], was then calculated:

[Γ/SA]=PEG surface density×πξ²

[Γ/SA]<1 indicates low PEG density where PEG molecules tend to be in the mushroom-like conformation, whereas [Γ/SA]>1 indicate high PEG density where PEG molecules tend to be in the brush-like conformation.^{3, 4} Estimations were shown in Table 1. Similar correlations between composition and configuration of surface conjugated PEG were reported by Wu et al. J Control Release 2011; 155:418-26.

High Resolution Multiple Particle Tracking

Human CVM was collected as previously described by Ward et al. J Magn Reson 2000; 143:79-87, following a protocol approved by the Johns Hopkins School of Medicine Institutional Review Board. Collected mucus samples were stored at 4° C. until used. Suspensions of fluorescently labeled liposomes were added at 3% v/v to human CVM (20 μL) for epi-fluorescence microscopy. Liposome transport rates were measured by analyzing trajectories of fluorescent liposomes, recorded using EM-CCD camera (Evolve 512; Photometrics, Tuscon, Ariz.) mounted on an Axio Observer epifluorescence microscope (Carl Zeiss AG, Oberkochen, Germany) equipped with a 100× oil-immersion objective (numerical aperture 1.46). Movies were captured using MetaMorph software (Molecular Devices, Inc., Sunnyvale, Calif.) at a temporal resolution of 66.7 ms for 20 s. Trajectories of n>100 liposomes were analyzed using customized MATLAB codes, and experiments in CVM from at least three different donors were performed for each condition. The coordinates of liposome centroids were transformed into time-averaged mean squared displacements (MSD), <Δr²(τ)>=[x(t+τ)−x(t)]²+[y(t+τ)−y(t)]² (τ=time scale or time lag), from which distributions of MSDs were calculated. The theoretical MSD of liposomes in water were calculated from MSD_{w}=4D_{w}τ, where D_{w} is the theoretical diffusivity of liposomes in water, and the time scale τ=1 s. Based on the Stokes-Einstein equation, D_{w}=k_{B}T/6πηR, where the Boltzmann constant k_{B}=1.38×10⁻²³ m²·kg·s⁻²·K⁻¹, T=293.15 K, the viscosity of water η=0.001 Pa·s, and R is the radius of the liposomes. The calculated theoretical MSD values are: 0 mol %-PEG, 3.3 μm²·s⁻¹; 3 mol %-PEG, 3.2 μm²·s⁻¹; 5 mol %-PEG, 3.5 μm²·s⁻¹; 7 mol %-PEG, 3.1 μm²·s⁻¹; 10 mol %-PEG, 2.9 μm²·s⁻¹; 12 mol %-PEG, 2.9 μm²·s⁻¹.

Chacterization of Liposomal Content and Retention of BA In Vitro

To characterize the content (i.e., agent:lipid ratio), BA-loaded liposomes were first freeze-dried, and further suspended in 10% v/v TRITON® X-100 solution. The encapsulated agent was then extracted by vigorous agitation of the suspended liposomes using a water bath sonicator. After centrifugation (21,000×g, 10 min), the supernatant was collected and further diluted in PBS. Fifty microliters of the diluent was injected into a Shimadzu high performance liquid chromatography (HPLC) system equipped with a c18 reverse phase column (5 μm, 4.6×250 mm, Varian Inc., Palo Alto, Calif.). BA was eluted using an gradient mobile phase [start with phase 1: water (100%), changing after 3 min to phase 2, water:acetonitrile (80%:20%, v/v)] and detected at 255 nm using a UV detector. Standard samples at known concentrations were first processed and calibration curves were generated as the reference for concentration calculations. Data were analyzed using LCsolution software (Shimadzu Scientific Instruments, Columbia, Md.). Drug:lipid ratio was defined as the weight ratio of encapsulated agents to the dried lipid components of the liposomes.

To characterize the retention of BA in the liposomes and the associated stability of the liposomal CEST contrast, 3 mL of newly prepared liposomes were instilled into a dialysis cassette (20 k Molecular Weight Cut Off, or MWCO, Thermo Scientific, Waltham, Mass.) and incubated in 200 mL PBS at 37° C. Dialysis was first performed to ensure all unloaded agents were eliminated. At pre-determined time intervals, 100 μl of liposome suspension was collected from the dialysate, followed by in vitro CEST imaging and HPLC measurement. For the latter, collected liposomes samples were further suspended in 10% v/v TRITON® X-100 solution and thoroughly agitated using a water bath sonicator, followed by centrifugation (21,000×g, 10 min). The amount of retained agents was then determined using HPLC as described above.

Animal Model

Naturally cycling, estrus phase female mice were used for the intravaginal distribution study and the in vivo CEST imaging studies. In brief, female CF-1 mice (6-8 weeks old, Harlan, Indianapolis, Ind.) were housed in a reversed light cycle facility (12-hour light/12-hour dark). Mice were selected for external estrus appearance, which was confirmed upon dissection. All animal studies were performed in accordance to protocols approved by the Institutional Animal Care and Use Committee (IACUC) at the Johns Hopkins University.

Intravaginal Distribution of Liposomes

Intravaginal distribution of liposomes was investigated via a method as previously described by Xu et al. J Control Release 2013; 170:279-86. For each formulation of liposomes, 10 μL of the liposomes (diluted 2× in water from stock suspension) was administered intravaginally. Within 10 min, vaginal tissues, including a “blank” tissue with no particles administered, were sliced open longitudinally and clamped between two glass slides sealed shut with superglue. This procedure completely flattens the tissue and exposes the folds. The blank tissue was used to assess background tissue fluorescence levels to ensure that all images taken were well above background levels. Five fluorescence images at 10× magnification were taken for each flattened vaginal tissue, and n=4 mice for each formulation tested. To quantify the uniformity of the fluorescence distribution, the variance-to-mean ratio (VMR) of the fluorescence was quantified using an approach similar to the conventional quadrant-based method (Nicholas et al. Biochim Biophys Acta 2000; 1463:167-78). In brief, each image was contrast-enhanced and normalized with 0.5% saturated pixels, then divided into 4×4 quadrants and the average fluorescence of each quadrat was determined using ImageJ (Bathesda, Md.). The VMR was defined as s²/x, where x and s represent the sample mean and standard deviation of the fluoresence intensities of the quadrats, respectively. For each formulation, the mean VMR was calculated by averaging the VMR values of all images (n≧15) collected from the corresponding group of mice. Lower VMR indicates lower variation of fluorescence intensity among quadrats, and thus more uniform distribution of the liposomes.

CEST Imaging In Vitro

All MRI were acquired at 310 K using an 11.7 T Bruker Avance system (Bruker Biosciences, Billerica, Mass.). The B₀ field was shimmed using the shimming toolbox in Paravision Version 5.1 (Bruker BioSpin MRI GmbH). A modified rapid acquisition with relaxation enhancement (RARE) sequence including a saturation pulse was used to acquire saturation images at different irradiation frequencies, which were used to generate the z-spectrum. A slice thickness of 1 mm was used, and the typical imaging parameters were: TE=4.3 ms, RARE factor=16, matrix size 128×64 mm and number of averages (NA)=2. The field of view was typically 13×13×1 mm on the number of phantoms. Two sets of saturation images were acquired, the first set consists of frequency map images for mapping of the spatial distribution of B₀, and the second set for characterization of the CEST properties. The acquisition time per frequency point was 12 s for frequency maps (TR=1.5 s) and 48 s for CEST images (TR=6.0 s).

For the B₀ frequency maps, WAter Saturation Shift Referencing (WASSR) was employed as described by Kim, et al. Water Saturation Shift Referencing (WASSR) for Chemical Exchange Saturation Transfer (CEST) Experiments. Mag. Res. Med. 2009; 61:14411450. A saturation pulse length (tₛₐₜ) of 500 ms, saturation field strength (B₁) of 0.5 μT (21.3 Hz) and a saturation frequency increment of 50 Hz (spectral resolution=0.1 ppm) was used for WASSR images. The image readout was kept identical between the frequency map images and CEST images. For CEST images, tₛₐₜ=4 s, B₁=4.7 μT (200 Hz), and a frequency increment of 0.2 ppm was used.

CEST Imaging In Vivo

Mice were anesthetized using isoflurane and positioned in a 11.7 T horizontal bore Bruker Biospec scanner (Bruker Biosciences, Billerica, Mass.). Twenty microliters of BA-loaded 7 mol %-PEG liposome suspension (4 mg BA/mL) or free BA solution at a equivalent dose were administered intravaginally via a customized catheter. Imaging was performed before and at 30 min-intervals after the intravaginal administration up to 1.5 h. Axial images were acquired at ˜2 mm above the tip of the catheter that was inserted ˜5 mm deep from the vaginal opening. CEST images were acquired through collection of two sets of saturation images, a WASSR set for B₀ mapping and a CEST data set for characterizing contrast. For the WASSR images, the saturation parameters were tₛₐₜ=500 ms, B₁=0.5 μT, TR=1.5 s with saturation offset incremented from −1 to +1 ppm with respect to water in 0.1 ppm steps, while for the CEST images, tₛₐₜ=3 s, B₁=4.7 μT, TR=5 s, with offset incremented from −6 to +6 ppm (0.2 ppm steps) with a fat suppression pulse. The acquisition parameters were: TR=5.0 s, effective TE=21.6 ms, RARE factor=12. T2-weighted images were acquired with TR=4.0 s, effective TE=32 ms and RARE factor=16.

Post Processing

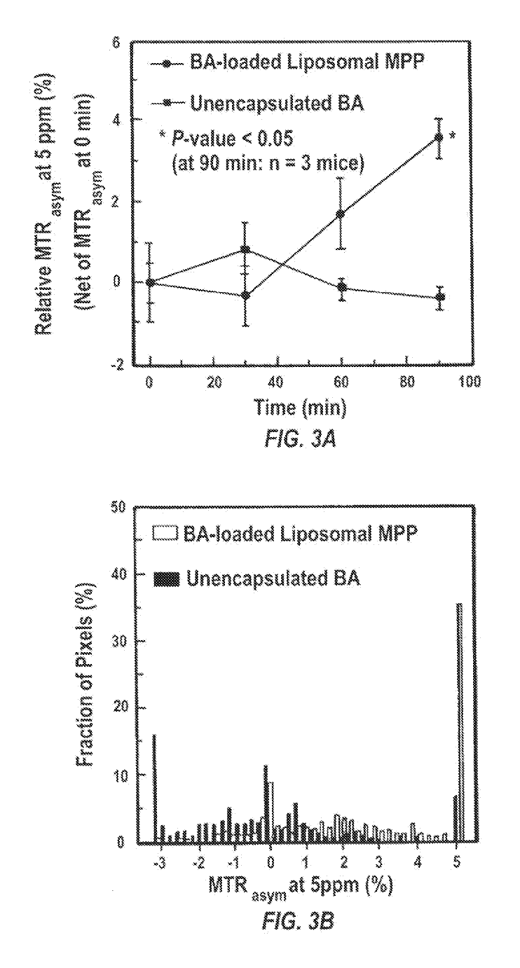

MR images were processed using custom-written Matlab scripts with the CEST contrast quantified by calculating the asymmetry in the magnetization transfer ratio (MTR_{asym}) using MTR_{asym}=(S_{-Δω}−S_{+Δω})/S₀ for NH protons at the frequency offset of BA from water (Δω)=5 ppm. S₀ is the signal of water without saturation, S with saturation and therefore frequency dependent. Time-lapse Relative MTR_{asym} was defined as the difference between MTR_{asym} values post-administration and pre-administration. Data in FIGS. 3A and 3B represent n=3 animals for each formulation group.

Statistical Analysis

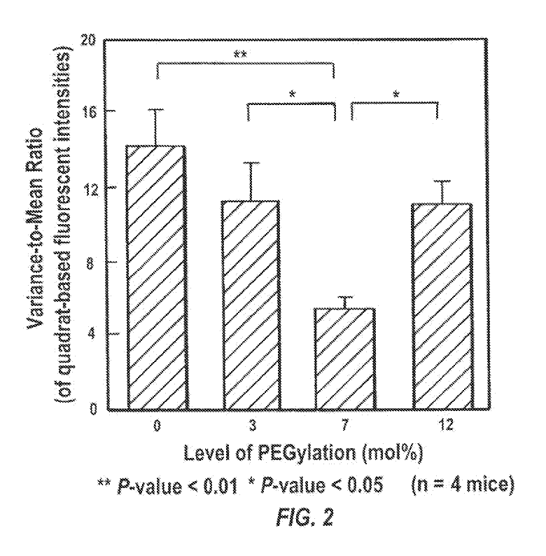

All data are presented as mean with standard error of the mean (SEM) indicated. Statistical significance of MSD between formulations (FIG. 3A; assuming log-normal distribution of MSD) was determined by one way analysis of variance (ANOVA) followed by a Tukey's test (homogeneous variance determined by a Levene's test). Differences in ID values between formulations (FIG. 2) were evaluated using a ANOVA followed by a Games-Howell test (heterogenous variance determined by a Levene's test). Statistical significance of Relative MTR_{asym} between formulations (FIG. 3B) was determined using a two-tail Student's t test (homogeneous variance determined by a F test). P-values<0.05 were considered statistically significant.

Results and Discussions

DSPC liposomes were formulated containing 6 different ratios of DSPE-PEG₂ₖ (Table 1). Extrusion was used to reduce the mean diameters of all formulations to below the average mesh size of human CVM (˜340 nm)⁴ to minimize steric hindrance. The PEGylated formulations were relatively uniform in size (low polydispersity index, or PDI), whereas non-PEGylated liposomes displayed high PDI, implying aggregation occurred. The actual molar fraction of DSPE-PEG₂ₖ was measured and the PEG surface density estimated. The Γ/SA ratios suggest that liposomes with ≧7 mol %-PEG were coated with brush-like PEG chains forming effective surface shielding, whereas those with ≦5 mol %-PEG were covered with mushroom-like PEG chains and, thus, less effectively shielded.

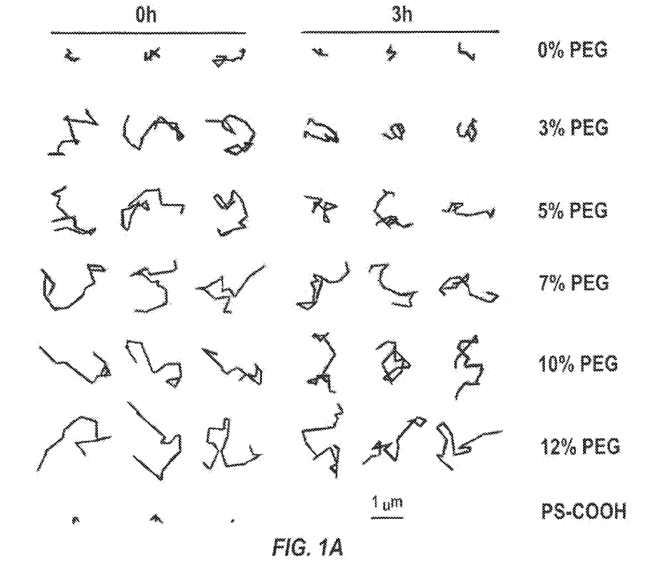

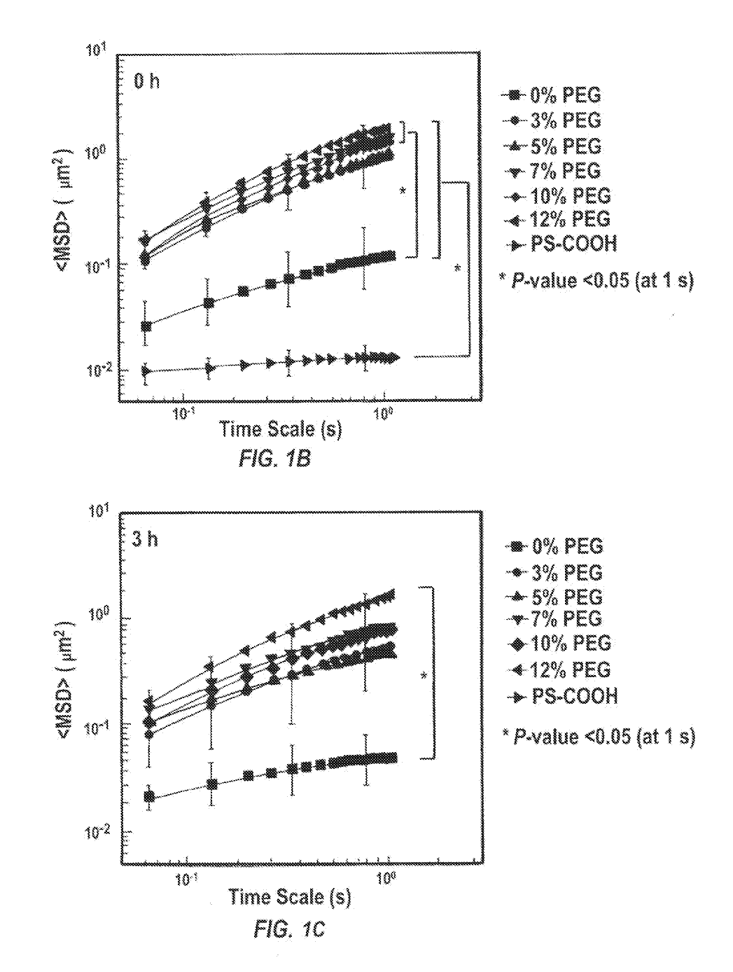

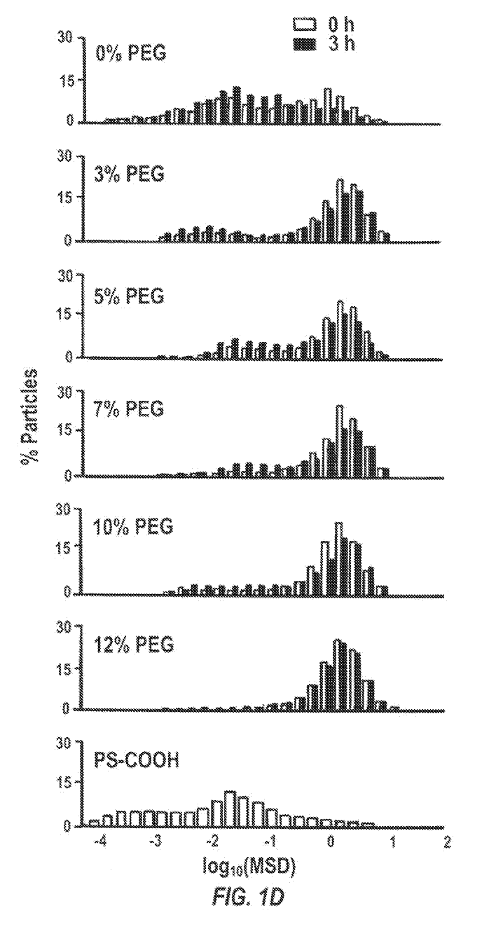

The diffusion of liposomes was calculated immediately (0 h) and 3 h after addition to CVM via multiple particle tracking (MPT). PEGylated liposomes diffused overall faster than the non-PEGylated liposomes, exhibiting more diffusive trajectories and ˜10-fold higher ensemble-averaged mean-squared displacement (<MSD>) (FIG. 1A). A significant population of immobilized non-PEGylated liposomes was revealed in the logarithmic distribution of individual liposome MSD (FIG. 1B, 1C). The <MSD> of PEGylated and non-PEGylated liposomes was ˜10- and 110-fold slower than their theoretical MSD in water (t=1 s), respectively (Table 1). After 3 h incubation in CVM, liposomes with lower PEG content (0-5 mol %) displayed more restricted trajectories and ˜2-fold decrease in <MSD>, with an increased immobilized fraction (FIG. 1D). Overall, liposomes with ≧7 mol % PEG diffused similarly in CVM compared to polymeric MPP (MSD_{w}/<MSD>ₘ˜10) and the mobility remained stable over time.

TABLE 1
Characterization of DSPC liposomes at different PEGylation
levels^{[a]}

| | | | Actual | PEG | | | |
|---|---|---|---|---|---|---|---|
| | Number | | Mol | Density | | | |
| | Mean | | % of | (Chains/ | | | |
| | Diameter | Polydispersity | DSPE- | 100 | [T/S | MSD_{w}/<MSD>ₘ^{[c]} | |
| Sample | (nm) | (PDI) | PEG₂ₖ | nm²) | A]^{[b]} | 0 h | 3 h |
| 0 mol %-PEG | 129 ± 18 | 0.37 ± 0.06 | NA | NA | NA | 110 | 270 |
| 3 mol %-PEG | 134 ± 9 | 0.09 ± 0.01 | 3.2 ± 0.1 | 7.2 | 0.6 | 13 | 25 |
| 5 mol %-PEG | 121 ± 9 | 0.06 ± 0.02 | 4.9 ± 0.1 | 10.9 | 0.9 | 14 | 31 |
| 7 mol %-PEG | 139 ± 4 | 0.06 ± 0.01 | 6.2 ± 0.1 | 13.9 | 1.2 | 8 | 15 |
| 10 mol %-PEG | 147 ± 9 | 0.03 ± 0.01 | 8.5 ± 0.2 | 18.8 | 1.6 | 8 | 15 |
| 12 mol %-PEG | 149 ± 5 | 0.04 ± 0.01 | 10.6 ± 0.3 | 23.7 | 2.0 | 6 | 7 |
| PS-COOH | 91 ± 1 | 0.04 ± 0.01 | NA | NA | NA | 1,400 | NA |

^{[a]}Containing 3-(trimethyl-silyl)-1-propanesulfonic acid sodium salt for NMR measurements.
^{[b]}Ratio of theoretical area covered by unconstrained PEG chains vs. total surface area of a liposome.
^{[c]}Ratio of theoretical MSD in water vs. <MSD> measured in CVM.

The prepared BA-loaded liposomes for diaCEST MRI are shown in Table 2. BA encapsulation minimally affected the liposome size and PDI. The loading capacity (BA:lipid ratio) correlated inversely with PEG content, with a significant drop at 12 mol %-PEG, likely due to reduced free volume associated with high PEG content on the inner surface of the liposomal shell, and the increased permeability of the lipid bilayer. The in vitro CEST contrast was generally consistent with the BA loading level.

TABLE 2
Characterization of BA-loaded liposomes

| | Number | | BA: | In vitro CEST |
|---|---|---|---|---|
| | Mean | | Lipid | Contrast |
| | Diameter | Polydispersity | Ratio | at 5 |
| Sample | (nm) | (PDI) | (%) | ppm (%) |
| 0 mol | 113 ± 12 | 0.28 ± 0.06 | 23 ± 4 | 32 ± 2 |
| %-PEG | | | | |
| 3 mol | 130 ± 5 | 0.05 ± 0.01 | 23 ± 3 | 28 ± 2 |
| %-PEG | | | | |
| 7 mol | 126 ± 7 | 0.06 ± 0.01 | 21 ± 1 | 21 ± 5 |
| %-PEG | | | | |
| 12 mol | 130 ± 3 | 0.08 ± 0.01 | 13 ± 4 | 13 ± 3 |
| %-PEG | | | | |

The vaginal distribution of BA-loaded liposomes in the vaginas of mice in the estrus phase of their estrous cycle were investigated. Particle mobility in mucus has been demonstrated to correlate with in vivo mucosal distribution (Ensign et al. Sci Transl Med 2012; 4:138ra79; Yang et al. Adv Healthc Mater 2013; Suk et al. J Control Release 2014; 178:8-17). Similarly, non-uniform distribution of mucoadhesive, non-PEGylated liposomes, was observed, which appeared to outline mucin bundles. This non-uniform distribution was also reflected by a high variance-to-mean ratio (VMR, increased VMR reflects decreased uniformity) (FIG. 2). While all PEGylated liposomes provided improved vaginal distribution, 7 mol %-PEG liposomes demonstrated the most uniform coverage with the lowest VMR. Additionally, individual cell outlines were observed, demonstrating that the 7 mol %-PEG liposomes were able to reach the vaginal epithelium. Liposomes with less PEG content may be insufficiently shielded to avoid mucoadhesion in vivo. Despite rapid diffusion in CVM, the 12 mol %-PEG liposomes also distributed suboptimally in vivo, perhaps due to their disassembly via micellization in vivo. Therefore, PEG content must be optimized to eliminate mucoadhesion while maintaining stability in vivo.

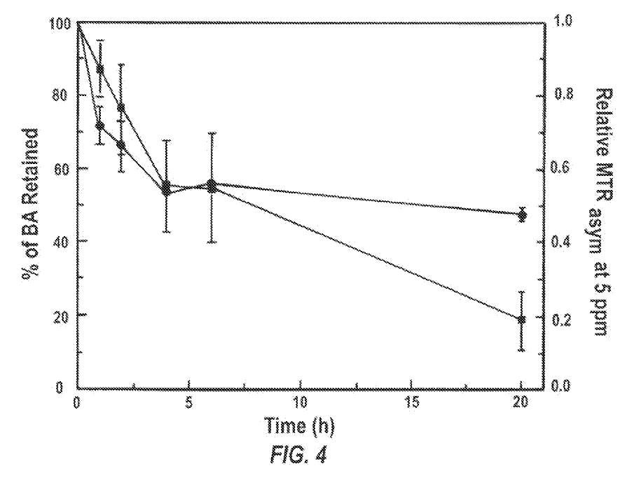

The vaginal retention of BA-loaded liposomes was monitored via diaCEST MRI. 7 mol %-PEG liposomes were selected as liposomal MPP given their sufficient loading and retention of BA (FIG. 4) and most uniform vaginal distribution. Liposomal MPP displayed good vaginal coverage with prolonged CEST contrast (at least 90min; highest relative MTR_{asym}^{5 ppm}˜4%); much shorter vaginal retention time was observed for unencapsulated BA (˜30 min; highest relative MTR_{asym}^{5 ppm}˜1%) (FIGS. 3A, 3B). The increase in CEST contrast over time for liposomal MPP was likely due to initial spreading throughout the vaginal tract, followed by liposome concentration at the epithelial surface as fluid was absorbed by the epithelium (FIG. 3B). At 90 min, images of liposomal MPP exhibited a significant fraction of high contrast pixels (MTR_{asym}^{5 ppm}˜5%) (FIG. 3B). CEST MRI has been previously used to monitor liposomes administered intratumorally and systemically. The results demonstrate the usefulness of diaCEST MRI for non-invasive monitoring of liposomes administered intravaginally. This capability could enable clinical evaluation of nano-carrier based vaginal therapies, especially when combined with new imaging methods.

In summary, liposomal MPP with optimized surface PEG shielding is capable of loading hydrophilic agents like BA. PEGylation, particularly at levels≧7 mol %, enhanced the mobility of liposomes in human CVM. However, increasing PEGylation to ˜12 mol % compromised drug encapsulation and in vivo distribution. Moderately PEGylated liposomes (˜7 mol %) maintained encapsulation efficiency while distributing most uniformly in the mouse vagina. Using non-invasive diaCEST MRI, it was shown that liposomal MPP provided uniform vaginal coverage and retained BA for ≧90 min in vivo. These results demonstrate the potential of liposomal MPP for mucosal delivery and imaging, and suggest that liposomal MPP formulations are suitable for theranostics in mucosal surfaces, like that of the vagina.

Modifications and variations of the pegylated liposomes for delivery of therapeutic, prophylactic or diagnostic agents to mucosal surfaces will be apparent to those from the foregoing descriptions and are intended to come within the scope of the following claims. The references cited herein are specifically incorporated herein.

1. A liposomal formulation, the liposome consisting of surface modified liposomes having a diameter of less than one micron, having enhanced mucosal penetration relative to liposomes that are not surface modified, having a molar ratio between surface modified lipid to non-surface modified lipid equivalent to between 3 and 11 mol % PEGylated liposomes to non-PEGylated liposomes.

2. The liposomal formulation of claim 1, wherein the liposomes contain from three to eleven mol % PEG-lipid to non-PEGylated-lipid.

3. The liposomal formulation of claim 2, wherein the liposomes contain 7 mol % PEG-lipid to non-PEGylated-lipid.

4. The liposomal formulation of claim 1, wherein the liposomes are modified with a neutral polymer selected from the group consisting of poloxamer (polyethylene glycol-polyethylene oxide block copolymers), poly(vinyl pyrrolidone) (PVP), poly(acryl amide) (PAA), and 1,2-Distearoyl-sn-glycero-3-phosphoethanolamine (DSPE) covalently linked to poly(2-methyl-2-oxazoline) or to poly(2-ethyl-2-oxazoline).

5. The liposomal formulation of claim 1, wherein the liposomes are modified with polyethylene glycol having a molecular weight of between 2000 and 5000 Daltons.

6. The liposomal formulation of claim 1, wherein the liposomes comprise a therapeutic, prophylactic or diagnostic agent.

7. The liposomal formulation of claim 1, comprising a phosphatidyl choline as the primary lipid.

8. A method for delivery of a therapeutic, prophylactic or diagnostic agent to a mucosal surface comprising administering the liposomal formulation of claim 1, wherein the liposomes comprise a therapeutic, prophylactic or diagnostic agent.

9. The method of claim 8 comprising administering the liposomes nasally, orally, vaginally, rectally, or pulmonarily.

10. The method of claim 8 comprising administering the liposomes onto or into the eye, or a compartment thereof.

11. The method of claim 8 wherein the liposomes are administered in a gel, ointment, lotion, emulsion, suspension, aerosol, or spray.

12. The liposomal formulation of claim 1, wherein the surface modified liposomes have a diameter of less than 500 nm.

13. The liposomal formulation of claim 7, wherein the phosphatidyl choline is 1,2-disteoroyl-sn-glycero-3-phosphatidylcholine (DSPC).
